(19) United States
(12) Patent Application Publication
(10) Pub. No.: US 2013/0028929 A1
(43) Pub. Date: Jan. 31, 2013

(54) FUNCTIONAL BEVERAGE COMPOSITION COMPRISING CHLORELLA AND DEEP SEA WATER

(71) Applicants: Mi Kyung Kim, Gyeongsangbuk-do (KR); Sung II Kim, Daegu (KR); Byung Hyuk Yoo, Daegu (KR)

(72) Inventors: Mi Kyung Kim, Gyeongsangbuk-do (KR); Sung II Kim, Daegu (KR); Byung Hyuk Yoo, Daegu (KR)

(73) Assignee: ECOPHYCO TECH CO., LTD., Kyongbuk (KR)

(21) Appl. No.: 13/640,909

(22) PCT Filed: Dec. 7, 2010
(86) PCT No.: PCT/KR10/08721

§ 371 Date: Oct. 12, 2012

(30) Foreign Application Priority Data
Apr. 13, 2010 (KR) 10-2010-0033980

(51) Int. Cl. A61K 36/05; A61P 39/06; A61P 35/00

(52) U.S. Cl. 424/195.17

(57) ABSTRACT

The present invention relates to a functional beverage composition comprising chlorella and deep sea water. The composition comprises 0.1-5 wt % of chlorella powder, 90-99 wt % of deep sea water, 0.001-0.5 wt % of a grapefruit extract, 0.01-5 wt % of honey, 0.01-5 wt of polydextrose, 0.001-1 wt % of citric acid, and 0.001-0.1 wt % of enzyme-treated stevia. Thus, the functional properties of deep sea water and chlorella, including antioxidant, anticancer, anti-inflammatory, cerebral vascular and cardiovascular disease inhibitory and anti-diabetic functions, can be provided by taking the beverage composition.

### TECHNICAL FIELD

The present invention relates to a functional beverage composition comprising chlorella and deep sea water.

### BACKGROUND ART

In recent years, the consumer's demands for favorite foods have been diversified, and due to a variety of stresses, the demand for foods beneficial to health has been increased. Thus, the beverage market is changing from chemical beverages to natural beverages.

According such consumer's demand, beverages having palatability and functionality, including corn silk tea, green tea beverages, deep sea water and vitamin beverages, are appearing in the market.

Among them, deep sea water is water deeper than 200 m below the sea surface, which has properties different from those of sea surface water. It has little or no organisms or pathogenic bacteria, is kept at a constant temperature throughout the year, is rich in nutrient salts essential for the growth of marine plants, and is sea water resources aged for a long period of time. In other words, deep sea water is useful marine resources having various characteristics, including low temperature, cleanness, eutrophication and aging properties. Also, it is vast clean resources, which is produced in the material circulation system using sunlight as an energy source and recycled as seawater.

Known technologies relating to beverages comprising such deep sea water are mainly technologies of preparing raw beverages by desalinating deep sea water.

Meanwhile, chlorella has various effects, including antioxidant, anticancer, anti-inflammatory, cerebral vascular and cardiovascular disease inhibitory and anti-diabetic effects, shows the effects of improving constitution and supplementing nutrients, and contains protein sources, chlorophyll, cellulose and the like. Thus, it is being used as a food raw material and a food additive.

Prior art documents that describe the use of chlorella include Korean Patent Application Nos. 1019830004343, 1019960004306, 1020017007429, 1020017003167, 1020010072416, 1020010054228, 1019950053339, and the like.

However, these prior technologies mainly use freshwater chlorella, and examples of freshwater chlorella which are distributed in Korea and other countries include Chlorella minutissima, C. pyrenoidosa, Chlorella vulgaris and the like. However, the application of marine chlorella species (photosynthetic green algae (microorganisms)) to beverages has not yet been reported.

In addition, a beverage based on deep sea water and marine chlorella has not been developed.

### DISCLOSURE

### Technical Problem

It is an object of the present invention to satisfy the consumer's demand for various health functional beverages and to provide a functional (antioxidant, anticancer, etc.) beverage comprising deep sea water and chlorella as active ingredients so that the active ingredients can be taken.

### Technical Solution

In order to accomplish the above object, the present invention provides a beverage composition comprising 0.1-5 wt % of chlorella powder, 90-99 wt % of deep sea water and 0.113-11.6 wt % of additives.

In the present invention, the additives include 0.001-0.5 wt % of a grapefruit extract, 0.01-5 wt % of honey, 0.01-5 wt % of polydextrose, 0.001-1 wt % of citric acid, and 0.001-0.1 wt % of enzyme-treated stevia.

### Advantageous Effects

According to the present invention, the natural pigments of marine chlorella (chlorophyll a and b, β-carotene, lutein, etc.) are added to deep sea water, and thus the functional properties of deep sea water and chlorella, including antioxidant, anticancer, anti-inflammatory, cerebral vascular and cardiovascular disease inhibitory and anti-diabetic functions, can be provided by taking the beverage composition comprising them. Also, various minerals abundant in deep sea water can be taken. The beverage composition of the present invention can contribute to health promotion.

In addition, flavonoids, pectin, potassium, vitamin C, folic acid and the like contained in grapefruits can also be taken, and the effect of improving blood circulation can be provided by the flavonoids.

### BEST MODE

Hereinafter, the present invention will be described in detail.

In one embodiment of the present invention, a functional beverage composition comprising marine chlorella and deep sea water comprises 1 wt % of chlorella powder, 94.55 wt % of deep sea water, 0.15 wt % of a grapefruit extract, 1.75 wt % of honey, 2.5 wt % of polydextrose, 0.04 wt % of citric acid, and 0.01 wt % of enzyme-treated stevia.

The marine chlorella that is used in the present invention is selected from among Chlorella sp., Chlorella ovalis, Chlorella saline and Chlorella marina. This chlorella contains natural pigments, including chlorophyll a and b, β-carotene, lutein and the like.

The chlorophyll has antioxidant, poison-neutralizing and anticancer effects, and the β-carotene is converted to vitamin A in the liver of the human or animal body, in which vitamin A functions to protect vision, normally maintain the eye mucosa, assist in recover from disease, promote growth, and assist in maintaining the skin, hair, bond and gum in a healthy state. If vitamin A is deficient, dry eye syndrome and nyctalopia can occur, gustatory and olfactory functions can be reduced, the skin become rough, and colds can be easily caught. Also, lutein is an antioxidant substance and absorbs reactive oxygen species that damage the macular rods and corns. It absorbs strong blue light that damages the macular rods and corns. Thus, it is particularly beneficial for eyes.

The deep sea water that is used in the present invention is used after desalination. The technology of desalinating the deep sea water is already known in the art, and thus the detailed description thereof will be omitted herein.

The grapefruit has low calorie and, at the same time, is rich in flavonoids, pectin, potassium, vitamin C, folic acid and the like. Particularly, naringin, a kind of flavonoid, assists in removing old erythrocytes and has the effects of controlling erythrocytic levels and preventing anemia. In addition, citric acid has a fatigue recovery effect, and pectin acts to lower blood cholesterol levels and has a good effect on the prevention of arteriosclerosis.

Further, the polydextrose that is used in the present invention is dietary fiber and is preferably used as a thickener for low-calorie food.

Citric acid assists in recovery from fatigue by removing lactic acid while it is metabolized in vivo. Specifically, citric acid acts to completely decompose lactic acid (that promotes the aging of cells or blood vessels and causes fatigue and various adverse effects such as allergy) into carbonic acid gas and water, which are then discharged from the body.

The enzyme-treated stevia is a natural sweetener.

As described above, the functional beverage composition comprising chlorella powder and deep sea water according to the present invention can exhibit various effects, including antioxidant and anticancer effects, when it is taken.

1. A functional beverage composition comprising marine chlorella and deep sea water, the composition comprising 0.1-5 wt % of chlorella powder, 90-99 wt % of deep sea water and 0.113-11.6 wt % of additives.

2. The functional beverage composition of claim 1, wherein the chlorella is one or more selected from among Chlorella ovalis, Chlorella salina and Chlorella marina.

3. The functional beverage composition of claim 1, wherein the additives include, based on the total weight of the composition, 0.001-0.5 wt % of a grapefruit extract, 0.01-5 wt % of honey, 0.01-5 wt of polydextrose, 0.001-1 wt % of citric acid, and 0.001-0.1 wt % of enzyme-treated stevia.
